(19) United States
(12) Patent Application Publication
(10) Pub. No.: US 2006/0015004 A1
(43) Pub. Date: Jan. 19, 2006

(54) Hemostatic device and methods

(76) Inventor: James Sitzmann, Zionsville, IN (US)

Correspondence Address: DAVID G. HENRY, 900 Washington Ave., P.O. Box 1470, Waco, TX, 76703-1470, US

(21) Appl. No.: 11/176,946

(22) Filed: Jul. 7, 2005

Related U.S. Application Data
(60) Provisional application No. 60/585,924, filed on Jul. 8, 2004.

(51) Int. Cl. A61F 13/00

(52) U.S. Cl. 600/37

(57) ABSTRACT

Methods and devices for hemostatic control of an injured internal organ. In one embodiment, a container is provided for at least partially surrounding an injured organ and exerting a compressive force upon the organ. Methods of treatment utilizing such devices are also provided.

### FIELD OF THE INVENTION

In certain embodiments, the present invention relates to medical devices for hemostasis and, in particular, devices and techniques related to inhibiting undesirable bleeding from an internal organ.

### BACKGROUND OF THE INVENTION

Hemorrhage from certain injuries to internal organs may present extremely challenging clinical scenarios. For instance, liver bleeding typically occurs after either massive abdominal trauma or from hepatic parenchyma rupture from a rapidly expanding tumor, for instance, from adenoma, hemangioma or hepatocellular cancer. Severe hemorrhage, with its associated hemodynamic instability, is an emergency situation that may result or cause an individual to go into shock. The situation may be further complicated by secondary physical injuries, as well as the development of secondary metabolic complications including coagulopathy, severe acidosis and hypothermia.

Known techniques for control of internal organ hemorrhage include hyperthermic coagulation, parenchymal mattress suture placement, vessel suture ligation, local procoagulant application and organ resection. For certain trauma patients, the use of pack or packing as part of a damage control laparotomy may be used in the treatment of a number of injuries. This technique allows the control of bleeding in a coagulopathic patient with very advanced injuries.

The successful management of liver trauma with the placement of abdominal packs was initially described by Feliciano, Mattox and Jordan, “Intra-abdominal Packing for Control of Hepatic Hemorrhage: A Reappraisal” J. Trauma 21(4):285-90 (1981). See also: Feliciano, Mattox, Burch, Bitondo and Jordan, “Packing for Control of Hepatic Hemorrhage” J. Trauma 26(8):738-43 (1986); Jacobson, Kirton and Gomez, “The Use of an Absoirbably Mesh Wrap in the Management of major Liver Injuries” Surgery 111(4):455-61 (1992). This technique has proven an extremely successful way to control liver bleeding from a major hepatic injury from trauma or following rupture of liver tumors.

Further, application of a procoagulant on, for instance, the raw liver surface, followed by firm packing with laparotomy sponges may also improve hemostatic control. Pack removal, which generally occurs once the patient is normothermic and not coagulopathic, approximately 24-48 hours after surgery, often causes clot disruption and excessive re-bleeding. This return of bleeding is particularly problematic when the initial injury involves, for instance, a large disruption of Glisson's capsule as a proportionately larger resultant rebleeding area will result.

The presently available devices and methods for hemostatic control all have certain inherent disadvantages, including, without limitation, the blood clot disruption problem described above. Particularly when extensive disruption of the liver surface has occurred, removal of directly applied packs, particularly with the coadministration of procoagulant, results in exfoliation and debridement of liver parenchyma with recurrent bleeding.

Accordingly, a need exists for devices and related methods for treating injury to internal organs so as to adequately stem undesirable bleeding and allow removal of packs without disturbance of the organ tissue and any clots associated with that tissue.

### SUMMARY OF THE INVENTION

One object of the present invention is to provide new devices and methods useful for treating damage to internal organs, and in particular, to reduce bleeding from damaged internal organs.

In one embodiment, the invention relates to a method of treating a larger mammal or human suffering from damage to an internal organ. The method includes the steps of at least partially surrounding the injured organ with a container and then exerting or applying a compressive force on at least a portion of the organ with the container. The application of the compressive force may be performed through inflation of one or more inflatable portions of the container or by packing the area around the container with sponges or other packing material.

In another embodiment, the invention relates to a device or system for treating a larger mammal or human suffering from damage to an internal organ. The device includes a container which may at least partially surround an organ and which is configured to exert a compressive force upon at least a part of the internal organ, using externally applied packing material and/or inflatable pouches or portions of the container, or another suitable compression means. The devise may also be provided with a procoagulant on or within the interior surface of the container.

### BRIEF DESCRIPTION OF THE DRAWINGS

FIG. 1 depicts a perspective view of a liver;

FIG. 2 depicts a perspective view of a container in accordance with one embodiment of the invention;

FIGS. 3a and 3b depict lateral views of containers in accordance with different embodiments of the invention;

FIG. 4 depicts a portion of the interior surface of a container in accordance with an embodiment of the invention;

FIGS. 5a and 5b depicts cross-sectional views of surface arrangements in accordance with certain embodiments of the invention;

FIG. 6 depicts a perspective view of a spleen;

FIG. 7 depicts a lateral view of a container in accordance with an embodiment of the invention; and

FIGS. 8a and 8b depict photographs of a device in accordance with the invention in operation.

### DETAILED DESCRIPTION OF PREFERRED EMBODIMENTS

The present invention may be understood by reference to the following detailed description of particular embodiments of the invention. The terminology used herein is for the purpose of describing particular embodiments only and is not intended to be limiting.

In one embodiment, the present invention provides a method of treating a patient, in particular a larger mammal or human, suffering from damage to an internal organ. In most instances, this damage will cause undesirable bleeding from that organ; however, there may be instances in which the methods of the present invention may be applied to injuries of another type. The method comprises the steps of at least partially surrounding the damaged organ with a container and then exerting a compressive force upon at least a portion of the damaged organ with the container. The container need not entirely surround the organ; however, in certain instances, it is preferable that the container almost or entirely surround the organ. The amount that the organ needs to be surrounded depends upon the particular damage to the organ and the resultant bleeding or other indications. For instance, if only a very discrete portion of the organ is damaged, it may be necessary to surround only a similarly discrete or slightly larger portion of the organ with the container. In this way, the compressive force may be applied only to that portion of the organ on or about which the bleeding is occurring.

A method for treating extensive organ rupture in a liver is described below; however, it should be appreciated by one of skill in the art that the following treatment method may be applied to a different organ, for instance, an injury to the spleen or another internal organ.

To treat extensive hepatic rupture, particularly if it involves the right hepatic lobe, it is generally preferable to perform a generous laparotomy, particularly extending the top of the incision to the xyphoid. In certain instances, the incision may be extended into the right chest through an anterior lateral trans-diaphragmatic incision. Further, in cases where significant liver disruption is noted, extensive liver mobilization, especially of the right hepatic lobe exposing the bare area of the liver, and supra-hepatic and infra-hepatic inferior vena cava, is performed. Further, division of the falciform ligament and the peritoneal attachments to the left lateral segment are performed. In order to optimally understand the liver injury and any associated vena caval injury, properly place the necessary liver sutures, achieve caval control, and, if required, to optimally provide circumferential hepatic tamponed packing it is useful to fully mobilize the right lobe of the liver.

If liver packing is necessary to treat extensive bleeding, coagulopathy and damage control, without caval injury or with repair of caval injury, the surgeon may begin by placing procoagulants such as gel foam and thrombin or surgicell on the raw liver surface. A Pringle maneuver, with or without vena caval control, may be used to decrease hepatic blood flow. Once the procoagulant is in place, the liver is “wrapped” by placing a sterile container around the liver. In certain instances, it may be desirable to place the container over the left and right lobes of the liver as far posteriorly as possible.

Depending on the construction of the container, it may also be folded under the liver. Packs, such as abdominal sponges, may then be placed firmly around the container, preferably in a clockwise fashion, from approximately 6-7 o'clock to 5 o'clock, to maintain pressure on the hepatic parenchyma and tamponade the bleeding. The packs may be placed systematically around the liver beginning in the posterior hepatic space. The patient's abdomen is then closed, utilizing a temporary abdominal closure technique. Dynamic abdominal retention may be a useful technique for this abdominal closure, as it is both rapid to perform and inexpensively preserves the abdominal domain.

Upon completion of the initial laparotomy, the patient may be taken to an intensive care unit for a period of approximately 48-72 hours in order to allow the liver to recover and for the reversal of hypothermia and coagulopathy. Following this period, the patient may be returned to the operating room, where the abdomen is opened and the packs are removed. The container is free from the liver and clot and may easily be removed from the liver surface without causing further bleeding. Depending upon the patient's condition, either primary closure or delayed secondary closure may be utilized.

The methods described have been successfully employed to treat an idiopathic hepatic rupture, a hepatic adenomatosis rupture, as well as hepatic ruptures caused by blunt trauma in motor vehicle accidents. Treatments resulting in proper healing and discharge for each of the patients so treated. The technique described herein provides the added benefit of easy and safe subsequent pack removal. Through the use of the container, there is no external adherence between the pack(s) and the clot or liver. This is particularly a useful technique when extensive disruption of the liver surface has occurred, because disturbance of the clot will likely result in recurrent bleeding. Procoagulants applied to the liver surface or on or within the container may greatly enhance the stoppage of bleeding. The ability of the liver to “regenerate” following parenchymal loss obviates concern for the possible loss of liver parenchyma from excessive packing pressure.

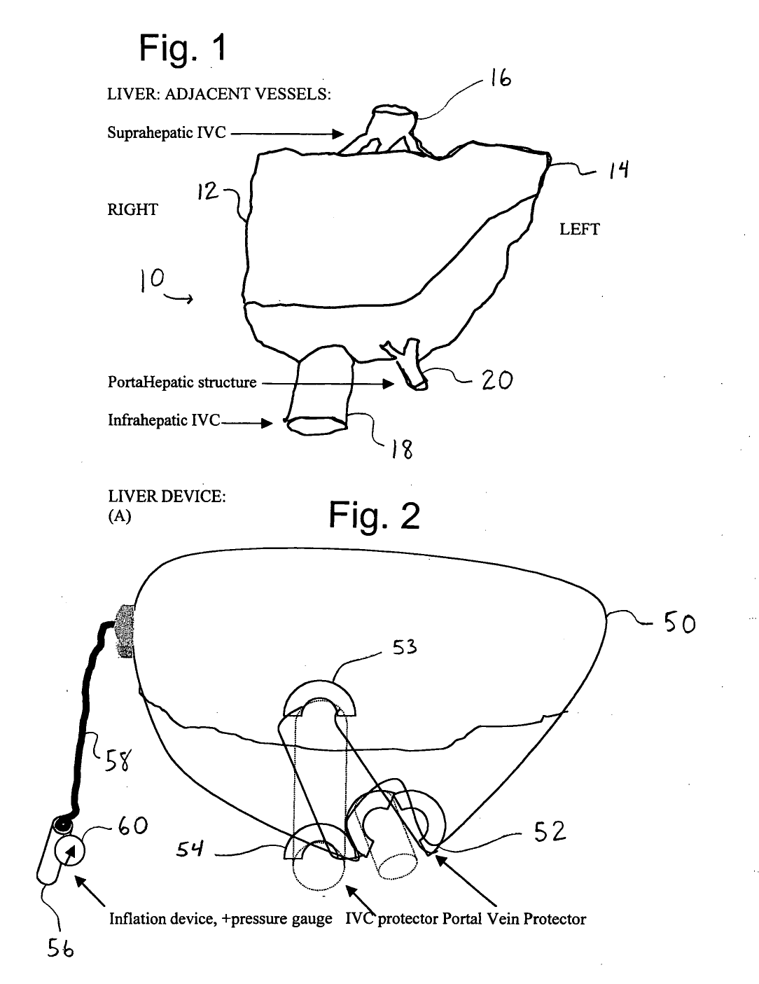

FIG. 1 depicts a perspective view of a liver 10. In FIG. 1, a liver 10 is shown with a right 12 and left 14 hepatic lobe. The suprahepatic inferior vena cava 16 is shown extending from the top of the liver 10 and the infrahepatic inferior vena cava 18 and porta hepatic structure 20 is show extending from the bottom of the liver 10.

FIG. 2 depicts a perspective view of a container 50 in accordance with one embodiment of the invention. In FIG. 2, a container 50 is shown that is configured to fold around the damaged or ruptured liver. The container 50 is generally shaped as an open bag which can fold around the liver to conform to the shape of the liver and allow mechanical packing with sponges, pads, packs or other packing material (referred to herein as pack(s)), or alternatively with air, as described below. The container 50 has a soft mold to conform to supra-hepatic vessels so as to avoid constriction or excessive compression. The soft mold is a preformed shape, that is generally not deformable. Preferably, the soft mold is deformable or compressible only with extreme force which could puncture the inflatable device. This level of force is not naturally present internally within mammals, and in particular, humans. In certain embodiments, the container 50 is provided in a generally C-shaped form to prevent infra or supra hepatic vena caval compression.

The container is C-shaped to prevent circumferentially encompassing and potentially enclosing the vena cava. The use of a C-shape prevents and inhibits the device from compressing the vena cava upon inflation of the device. In fact, the inflation typically leads to a raising or lifting of the liver from the retro-peritoneum. This minimizes the gravitational weight effect on the vena cava for a supine patient, which may sometimes result from intrahepatic blood adding to the weight of the organ.

The container 50 may be constructed from any suitable material including polyethylene, polypropylene, polyurethane, silastic, silicon or Teflon. Preferably, the container is constructed as a single unit with the inflatable gauge attached to the tube after implantation. Alternatively, the container may be constructed from multiple separate pieces. The material for the container may be either monolayer or a bi or trilayer. The latter material is especially useful if one or more bioactive coagulant or sealants are provided.

For surgical applications preferably the container is provided in a sterilized packaged form. In particular, it is preferably that there are no allergenic animal proteins, silicone, and/or no use of any latex in the packaging or any element of the container or its internal and external parts (for instance the pressure gauge).

Preferably, the device is easily manufactured and easily stored in a sterile bag which may placed in a stackable, low profile box. The pressure gauge used for inflation may also be provided in the same packaging. In operation, the pressure gauge may be connected to the inflation tube after placement or installation of the device. Preferably, the pressure gauge exits the tube out the patient's flank. The sealants may be placed or stored in a separate container or storage box and added at or near the operative field to the inflatable container device.

Further, the container 50 may be provided with one or more locks 52, 53 and 54 to form circumferential collars around porta-hepatic vessels and structures such as the portal vein, hepatic artery and bile duct or gallbladder. Preferably, these locks 52, 53 and 54 are soft, i.e. they do not have a rigid shape, and may, for instance, be constructed from a suitable plastic or Velcro or a combination thereof.

The purpose of the two locks is to prevent vascular compression and blood flow reduction. Preferably, one lock is a soft collar and is provided with a 75%-90% circumferential protective semi rigid balloon attached to the whole device so that it may maintain its shape during and after inflation, preferably even under external pressure. In operation, this soft collar will maintain its circular, protective shape and thereby provide a protected space or area. Another lock may be provided with a 100% circular shape. This lock is preferably made with a hard material (e.g., nylon or plastic) which is not inflatable and not compressible. This hard lock is attached to the inflatable container device. The hard lock is provided with an opening that is opposite a hinge so that the hard lock may be opened and closed. Preferably, the hard lock is configured for positioning around the porta hepatus (which includes the following anatomic structures: hepatic artery, portal vein, bile duct and cystic gall bladder duct).

In one embodiment, the container 50 is provided with one or more inflatable chambers. These inflatable chambers are such that when the container 50 is placed around or beside the liver, or other injured organ, the container 50 may exert or apply a force against and partially compress part of the liver or other injured organ. For purposes of the present disclosure, the term compressive force is used to refer to any force which is applied against the outer surface of the organ. Thus, the compressive force may be exerted in multiple directions, especially where, for instance, the container 50 at least partially surrounds the organ being treated.

In the embodiment where the container 50 includes one or more inflatable chambers, the container 50 is preferably provided with an inflation device 56. The inflation device 56 may be an automatic or manual pump to pump air into the container 50 through an inflation tube 58 such as an air hose or other suitable air delivery apparatus. In a preferred embodiment, the inflation tube 58 extends through the patient's skin through an incision so that the pressure of the air within the chamber, and the related compressive force exerted by the container 50 may be adjusted up or down. The inflation device 56 may alternatively be a coupling to connect to an available air supply such as from an external air pump or pressurized canister. In this regard, the inflation device 56 may be a coupling provided with a valve, luer lock or needle valve. In yet another alternative, the inflation device 56 may be a syringe, which may optionally be provided with a valve, luer lock or needle valve. Further, the inflation device 56 may be provided with a pressure gauge 60 to provide an indication of the inflation pressure within the container 50.

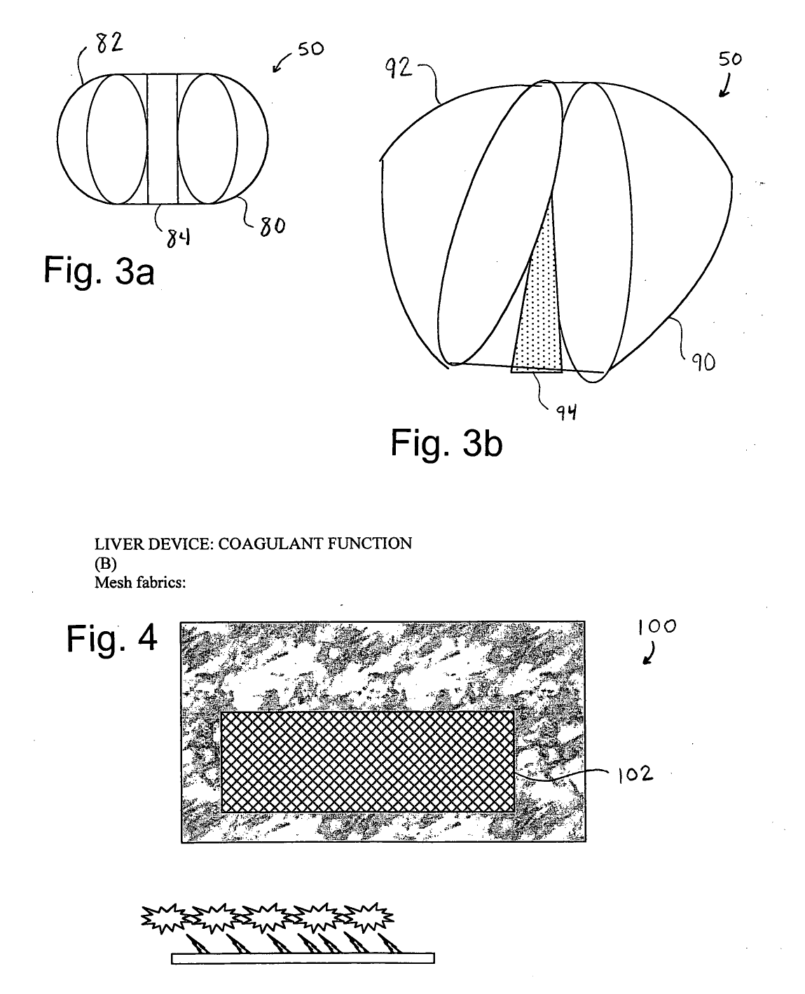

FIGS. 3a and 3b depict lateral views of a container 50 in accordance with different embodiments of the invention. In the container 50 shown in FIG. 3a, two lobes 80 and 82 are shown, one for each of the left and right lobes of the liver or other organ to be held within said the container 50. An optional spacer segment 84 is also shown separating the two lobes 80 and 82. FIG. 3b shows another container 50, this one also having two lobes 90 and 92, however the spacer segment 94 is triangular so that the two lobes 90 and 92 are nearer one another at the narrow end of the spacer segment 94.

FIG. 4 depicts a portion 100 of the interior surface of a container in accordance with an embodiment of the invention. In the portion 100 of the interior surface shown, the fabric or material from which the container is formed is impregnated or coated with a procoagulant 102 such as fibrin or thrombin. Alternatively the procoagulant or sealant may be provided along with or substituted by a cellulose material, or a separate biodegradable material including the procoagulant may be provided as at least part of the inner surface of the container.

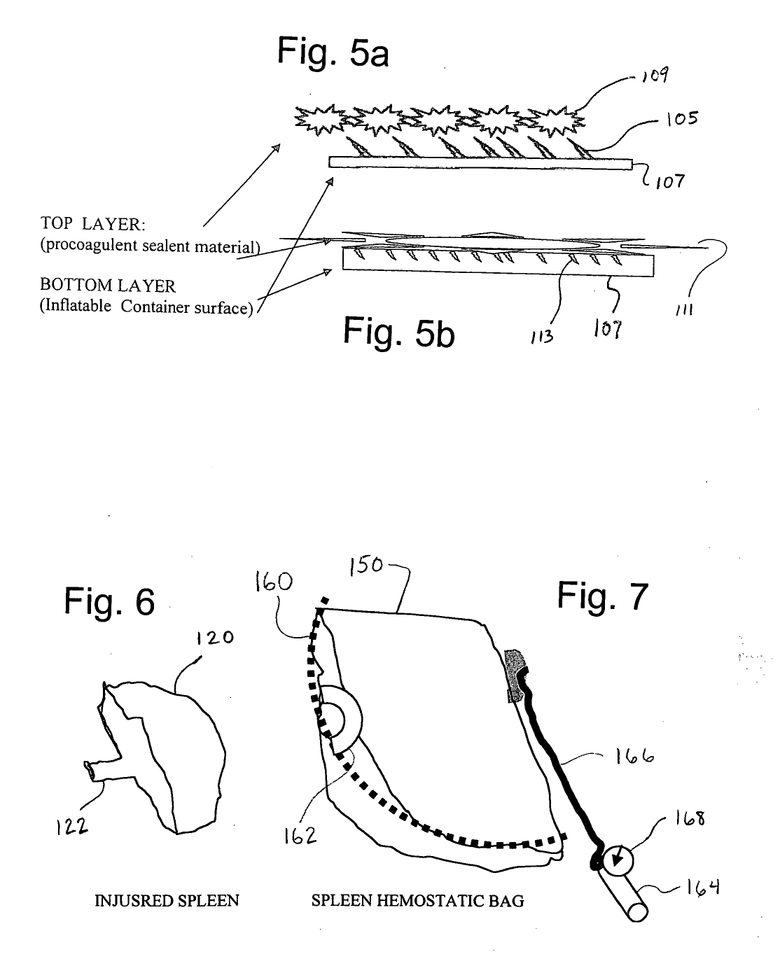

FIGS. 5a and 5b show embodiments of a multilayer surface which is preferably used with a biodegradable pro-coagulant material which abuts the surface of the organ being treated.

FIG. 5a shows an embodiment of the surface of a bilayer or trilayer attachment for the surface of the container device. In particular, soft or flexible spikes or barbs 105 are distributed on the surface 107 of the container to attract and also to grasp and release biologic (for instance, fibrin or collagen) or other pro-coagulant sealant material 109. In a preferred embodiment, these spikes or barbs 105 are distributed sparsely on the surface 107. A preferred spike or barb distribution is in the range from 10 to 1,000 per cm². Preferably the spikes or barbs 105 are from approximately 2-7 microns in length and approximately 1-3 microns in diameter. The relatively small scale of the spikes or barbs 105 permits their use and the removal of the container without damage, debridement or retraction of the clot on the surface of the liver or other organ being treated. Similarly the small scale avoids direct abrasion of the surface of the organ being treated.

FIG. 5b shows another embodiment of a surface similar to that shown in FIG. 5a for holding the biologic or other pro-coagulant sealant material 111. In the surface shown in FIG. 5a, hollow surface internal spikes or pores 113 are provided. These internal spikes or pores 113 can be at least partially filled with an absorbent and preferably rapidly biodegradable glue or other adhesive to provide attachment to the pro-coagulant or sealant layer. Preferably the pro-coagulant or sealant layer is also biodegradable.

The pro-coagulant or sealant layer is preferably easily released from the container surface, and the surface preferably does not debride, damage, or retract the clot from the surface of the liver or other organ being treated upon removal. In many instances the surface of FIG. 5b with the internal holes may have better release qualities than the surface shown for FIG. 5a.

In another embodiment (not shown) the container is configured from a monolayer and has a flat smooth planar surface with preferably less than approximately 0.5 micron irregularity or undulation. Further, this surface is preferably non-porous.

In one method, packs are provided with biological procoagulant sealants. These packs are provided around the organ being treated so that the biological procoagulant sealants directly abut the organ being treated. The container may then be placed around the combination of the organ being treated and the packs. In such a method the container may not require any preattached coagulant or sealant, and may, for instance be configures from a monolayer as described above.

FIG. 6 depicts a perspective view of a spleen 120. In FIG. 6, a spleen 120 is shown with a spleenic artery and vein 122 entering from one side of the spleen 120.

FIG. 7 depicts a perspective view of a container 150 in accordance with another embodiment of the invention. In FIG. 7, a container 150 is shown that is configured to fold or at least partially surround the damaged or ruptured spleen. The container 150 is generally shaped as an open bag with a soft mold, similar to that described above, so as to avoid undesirable compression or constriction of spleenic vessels. The container 150 may be configured from any suitable material such as polyethylene, polypropylene, polyurethane, silastic, silicone or a Teflon material.

In practice, the container 150 is placed around the spleen and then it may be shut using certain closing features 160 which are provided as part of the container 150. The closing features 160 may include any variety of suitable devices to close or seal a bag-like structure, such as plastic adhesive or Velcro.

The container 150 may also be provided with a lock 162 which forms a circumferential collar to go at least partially around the spleenic vessels and pancreas. The lock 162 is preferably soft and may be constructed from any suitable material including, for instance, plastic, Velcro or the lock 162 may be inflatable.

Preferably, the container 150 is provided with one or more inflatable chambers similar to that described above with respect to FIG. 2. Inflation of the one or more chambers causes the exertion of a compressive force upon the spleen. This works, at least in part, because the spleen is within a closed environment.

Much like the container 50 described with respect to FIG. 2, the container 150 of FIG. 7 may be provided with an inflation device 164 which may include a pump or syringe for forcing air, or another suitable fluid material through a hose 166 and into the one or more inflatable chambers. Preferably, the inflation device 164 is constructed so that the pressure within the inflation system and inflatable chambers may be adjusted and increased or decreased as desired.

Further, a pressure gauge 168 may be provided with, or as part of, the inflation device 164 to facilitate monitoring of the pressure within the inflation system.

A procoagulant may be provided directly against the spleen surface or it may be provided on or as part of the container 150. Suitable materials serving as carriers for the procoagulant may include fibrin, cellulose, a biodegradable fabric or mesh material impregnated or coated with fibrin or thrombin.

In certain embodiments, the container 150 is constructed so that it may be installed either during open laparotomy or with a laparoscope. The basic container with collars is a single piece which may be rolled and inserted through a standard large bore trocar. A reinforced edge or portion is provided to grasp the container with laparoscopic forceps and not tear, rip or perforate the container as it is placed in position. The placement may be without or after the placement or application of any desired pro-coagulant or sealant material. After placement, the collar may be snapped shut and the inflation tube exited trans-cutaneously out the patient (preferably through the flani) and then manually attached to the inflation gauge. A syringe or other inflation device may then be attached to the gauge and used to inflate the device, under vision provided via the laparoscope, noting the pressure achieved on the gauge.

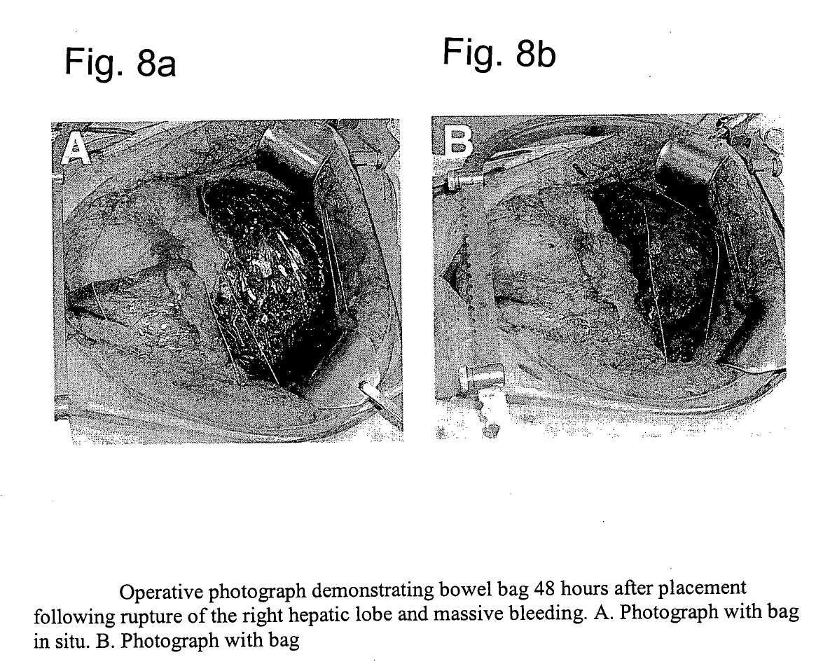

FIGS. 8a and 8b show operative photographs demonstrating the bowel bag 48 hours after placement following rupture of the right hepatic lobe and massive bleeding. FIG. 8a depicts an anterior superior view of the bag placement before sponge pack placement. FIG. 8b is a corresponding view after sponge pack placement. The plastic “bowel bag” was used to place circumferentially around 100% of peritoneal (exposed) liver surface area. The only part not covered is the retroperitoneal vena cava portion, as all other ligamentous attachments (falciform ligament, diaphragmatic ligament, hepato-gastric ligament, and right posterior ‘gutter’ retroperitoneal ligament) were dissected free, to mobilize and expose the liver for packing placement, after covering the liver with the plastic ‘drape’.

The container 150 may be provided in a variety of different sizes, so as to conform to the various sizes of spleens that may be encountered. For human patients, for instance, 5 sizes may be provided, grouped in accordance with the weight of the patient. These sizes may be grouped for 5-20 lb patients, 20-80 lb patients, 80-120 lb patients, 120-180 lb patients and 180-300 lb patients.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A method of treating a larger mammal or human suffering from damage to an internal organ, comprising the steps of:
at least partially surrounding said organ with a container, and
applying a compressive force upon at least a portion of said organ with said container.

2. The method of claim 1, wherein said compressive force is exerted using a combination of said container and one or more sponges which may be placed around said container.

3. The method of claim 1, wherein said container includes at least one inflatable portion, said inflatable portion being configured to exert a compressive force upon said organ when inflated.

4. The method of claim 1, wherein said container at least partially surrounds at least a portion of said organ.

5. The method of claim 1, wherein said container substantially surrounds at least a portion of said organ.

6. The method of claim 1, wherein a procoagulant is applied to said organ.

7. The method of claim 6, wherein said procoagulant is either fibrin or thrombin.

8. The method of claim 1, wherein a procoagulant is provided on an interior surface of said container.

9. The method of claim 1, wherein a procoagulant is provided with a biodegradable material.

10. The method of claim 1, wherein said organ is a liver or spleen.

11. The method of claim 1, wherein said compressive force is applied for a period of from about 48 to about 72 hours, after which period said container is removed.

12. The method of claim 1, wherein said damage to an internal organ comprises damage which results in bleeding or hemorrhaging.

13. A device for treating a larger mammal or human suffering from damage to an internal organ, said device comprising:
a container which at least partially surrounds said internal organ and which is configured to exert a compressive force upon at least a part of said internal organ.

14. The device of claim 13, wherein said container is constructed from a flexible material which is suitable for insertion in the abdomen of a larger mammal or human.

15. The device of claim 13, further comprising one or more sponges which are placed around said container, and said container and said sponges are operative to exert a compressive force upon said organ.

16. The device of claim 13, wherein said container comprises at least one inflatable portion which may be used to exert a compressive force upon said organ.

17. The device of claim 16, wherein said at least one inflatable portion of said container is provided with an inflation device.

18. The device of claim 17, wherein said inflation device is configured to facilitate adjustment of the pressure provided within said inflatable portion of said container.

19. The device of claim 13, wherein said container is configured with a closure device to at least partially close said container.

20. The device of claim 13, wherein said container may be securely closed with an adhesive or Velcro.

21. The device of claim 13, wherein said container is configured to conform to one or more ducts or blood vessels so as to avoid compression of said one or more ducts or blood vessels.

22. The device of claim 13, wherein said container further comprises one or more locks which serve as a collar to at least partially surround one or more ducts or blood vessels.

23. The device of claim 13, wherein said container further comprises a procoagulant.

24. The device of claim 23, wherein said container further comprises a biodegradable material to deliver said procoagulant.

25. The device of claim 23, wherein said procoagulant is provided on an interior surface of the container.
